(19) United States
(12) Patent Application Publication
(10) Pub. No.: US 2026/0079108 A1
(43) Pub. Date: Mar. 19, 2026

(54) LABEL-FREE IDENTIFICATION OF TUMOR TISSUES BY COHERENT NONLINEAR VIBRATIONAL MODE IMAGING

(71) Applicant: THE REGENTS OF THE UNIVERSITY OF CALIFORNIA, Oakland, CA (US)

(72) Inventors: Wei XIONG, La Jolla, CA (US); Bin YANG, La Jolla, CA (US); Jianyu REN, La Jolla, CA (US); Chun-Chieh YU, La Jolla, CA (US); Jackson C. WAGNER, La Jolla, CA (US); Zishan WU, La Jolla, CA (US)

(21) Appl. No.: 18/887,926

(22) Filed: Sep. 17, 2024

(51) Int. Cl. G01N 21/63; G01J 3/433; G01N 21/3563; G01N 21/359; G01N 21/39; G01N 33/483; G02B 21/34; G02B 21/36

(52) U.S. Cl. CPC G01N 21/636; G01J 3/4338; G01N 21/3563; G01N 21/359; G01N 21/39; G01N 33/4833; G02B 21/34; G02B 21/361; G01N 2333/78

(57) ABSTRACT

A multimodal hyperspectral vibrational sum-frequency generation (VSFG) platform and a method of tumor diagnosis are provided. The method employs the chemical-specific VSFG microscopy platform as a label-free imaging technique for tumor identification.

### GOVERNMENT RIGHTS

This invention was made with government support under Grant Number 1R35GM138092-01 awarded by the National Institutes of Health, and Grant Number CHE-1828666 awarded by the National Science Foundation. The Government has certain rights in the invention.

### FIELD OF THE INVENTION

The present invention relates to a hyperspectral chemical imaging platform using line-scanning Vibrational Sum-frequency Generation (VSFG) microscopy. The platform and method can be applied in biomedical imaging, such as label-free imaging technique for tumor identification, and diagnostic modality for oncology.

### BACKGROUND

Vibrational sum-frequency generation (VSFG), a second-order nonlinear optical technique, has been used extensively as a spectroscopy tool to chemically profile symmetry-allowed samples. Traditionally, VSFG has been applied to interfacial systems (i.e., gas-liquid, liquid-liquid, gas-solid, solid-liquid), which lack inversion symmetry—a requirement for VSFG activity. This application of VSFG has provided a wealth of molecular details of buried interfaces, configurations of water molecules at interfaces, and chemical species at interfaces. Although VSFG has been powerful in determining molecular species and configurations at interfaces, its potential in measuring molecular structures of materials lacking inversion centers has not been fulfilled. This is partly because the materials could be heterogeneous in their chemical environment, compositions, and geometric arrangement, and a traditional VSFG spectrometer has a large illumination area on the order of 100 μm. Thus, traditional VSFG spectroscopy reports on ensemble-averaged information of the sample over a typical 100 μm illumination area. This ensemble averaging may lead to signal cancellations between well-ordered domains with opposite orientations and mischaracterization of local heterogeneities. The traditional VSFG is limited in measuring molecular structures in biomedical research.

Biomedical imaging tests and biopsy imaging constitute a major pillar of the clinical management of many human diseases including cancer, central to diagnosis, staging, as well as predicting and evaluating therapeutic response. Traditional histopathology relies on tissue staining with dyes or probes (including antibodies) that label specific tissue, cellular, and molecular components. Recently, advanced and multimodal imaging approaches are being developed to achieve sensitive, multiplexed and high-performance biomarker detection, as a new promise for improved precision and accuracy of tumor detection and a better cancer care. Despite their wide clinical and research applications, use of colorimetric or fluorescent probes to label and image tissue components often requires time-consuming tissue fixation and staining processes. It is highly limited in multiplexed detection, and the detection depends on the target abundance as well as the availability and specificity of probes. In contrast, label-free imaging offers high chemical sensitivity and preserves sample integrity by directly probing chemical or structural changes in biomarkers, such as proteins, DNA/RNA and lipids in tumor tissues. It also requires simpler sample processing and is not limited by the development of new detection probes. These advantages make label-free imaging a robust standalone detection method as well as a highly compatible module to be integrated into high-plex models of digital pathology. Therefore, it is desirable to develop a system and method for the label-free imaging technique for cancer diagnosis.

### BRIEF SUMMARY

The disclosure provides a multimodal nonlinear hyperspectral VSFG microscope platform for chemical imaging and analysis, and a method of label-free identification of tumor tissues. The platform employs a pulsed laser beam; an optical parametric amplifier (OPA), configured to guide the output of the laser beam, generating a mid-infrared (MIR) beam; a Fabri-Perot etalon, configured to pass the residue laser beam from the OPA for an up-conversion near IR (NIR) beam; a dichroic mirror (DM) that is transmissive to the MIR and reflective to the NIR; a resonant beam scanner mounted to an integrated 2-position slider (I2PS); an inverted microscope configured to integrate with the resonant beam scanner and to direct the overlapped two beams into the inverted microscope; a microscope stage for retaining a sample, to allow the two overlapped beams to focus onto the sample with a purely reflective Schwarzschild objective (SO); an infinity-corrected refractive objective (RO), collecting a VSFG signal generated by the sample; a linear polarizer and a telecentric tube lens system, where the signal is guided through and processed; a monochromator (MC), wherein a magnified image formed at the entrance slit of the MC; and a spectrograph with a charge-coupled device (CCD) attached with the linear polarizer and the telecentric tube lens system, where a magnified image and data are detected and collected.

In one aspect, the method for detecting tumor in a biological tissue sample with the VSFG microscope platform is disclosed herein. The platform and method provide chemically specific domain details of collagen in the biological tissue samples, enabling the detection of tumor and diagnostics of cancer as the label-free techniques.

The method includes the steps of mounting the tissue sample on a slide onto the microscope stage; subjecting the tissue sample with the NIR and MIR beams using Objective 1 (OL1) of the microscope; turning on the resonant beam scanner to raster the laser beams on the tissue samples; collecting a resulting VSFG signal using Objective 2 (OL2) of the microscope; directing the VSFG signal to a spectrometer and a CCD for data collection and processing. The additional steps include obtaining VSFG hyperspectral images using the resonating line scanner; and analyzing spectral signatures of the images for the NH_{S}/CH_{2,Ss} and CH_{S}/CH_{2,Ss} ratio mapping that directly differentiate between tumor and healthy tissues in the biological sample.

In one aspect, the biological tissues contain tumor related collagen.

In another aspect, the diagnostic method can be used in other types of tumors, such as lung tumor, breast cancer, colorectal liver metastases, esophageal squamous cell carcinoma, or malignant ovarian neoplasms.

### DESCRIPTION OF THE DRAWINGS

FIG. 1 provides a 3D schematic of an exemplary multimodal hyperspectral VSFG microscope platform according to an embodiment of the inventive system. Panel a is a top view of the core setup. A pump laser was sent to an optical parametric amplifier (OPA) to generate a tunable mid-IR pulse. The residual laser beam was frequently narrowed by an etalon (E) and spatially filtered into a Gaussian beam by a spatial filter (SF). Mid-IR and 1025 nm beams are spatially overlapped at a customized dichroic mirror (DM) and guided through the inverted microscope (boxed region in Panel a. Panel b illustrates two beams that are sent to a resonant beam scanner mounted to an integrated 2-position slider (I2PS), enabling seamless switching between brightfield and nonlinear optical modalities. The microscope platform is equipped with a reflective-based infinity-corrected Schwarzschild objective (SO) acting as a condenser and a refractive-based infinity-corrected imaging objective (RO) mounted to a vertical nanopositioning (VNP) z-axis stage. The SO focuses the line of incoming beams that the resonant beam scanner reflects onto the sample while the RO collects the VSFG line section of signals. It is preferable to fine-control the z-axis position of the RO at 1 μm precision to ensure the sample is at the best focal condition for high-quality imaging. The collimated line of the VSFG signal is then directed to a tube lens system composed of two tub lenses (TL1 and TL2), forming a line of magnified image at the entrance slit of the monochromator (MC). This line of image signal is then dispersed into frequency-resolved spectra by the MC, and then hyperspectrally imaged on a CCD. After collecting hyperspectral lines as a CCD image, the sample is scanned in the axis perpendicular to the resonant beam scanner scanning axis using the NP. To collect bright field images of the sample, the I2PS is moved to the brightfield position, and a mirror intercepting the white light source (WLS) is installed. Light is then focused by the RO and imaged by the SO. An image is then formed at the sensor plane of the brightfield camera (BC) at the top of the inverted microscope. Panel c provides a detailed view of the optical path through the tube-lens area into the MC and CCD. Panel d provides a detailed view of the sample area between the SO and RO.

FIG. 2 shows a representative image quality for rough alignment of brightfield imaging modality of a ZnO pattern.

FIGS. 3A-3D provide the vertical axis calibration workflow showing how to convert the CCD pixels to vertical spatial dimensions in the unit of μm. FIG. 3A is an image of the ZnO patterned coverslip. The small vertical bar (25 μm) represents the pixel distance from one to other edges of the pattern). Because the ZnO pattern cross is designed to have a 25 μm width, one can use the ratio of physical width to pixel width here to calculate the physical/pixel dimension ratio. FIG. 3B provides a representative, vertical axis calibrated image. FIG. 3C plots the dimensional of vertical slice. FIG. 3D plots the derivative of the vertical slice is taken to obtain the spatial resolution.

FIGS. 4A-4C is a diagram of an exemplary experimental design according to an embodiment of the invention of the VSFG microscope platform to image collagen tissues. FIG. 4A is a schematic diagram of collagen structure changes in tumors. The top figures indicate the normal collagen structure, while the bottom figures illustrate the possible structural changes caused by a tumor. From left to right, the columns represent changes in distance, diameter, and orientation, respectively. FIG. 4B diagrammatically illustrates the difference in optical signals toward collagen arrangement: VSFG, as a coherent nonlinear optical signal, can exhibit sensitivity to fibril/fiber packing; in contrast, incoherent optical signal, such as fluorescence, is insensitive to these arrangements. FIG. 4C is a schematic diagram of VSFG microscopy and the corresponding fiber and lab frame (inset). Jablonski diagram of VSFG (right). WP: wave plate; DM: dichroic mirror; GM: gold mirror; SM: silver mirror; OL: objective; PR: Polarizer; TLs: Tube Lens.

FIGS. 5A-5D illustrate mechanical and IR mapping of collagen tissues, where FIG. 5A shows AFM imaging of the collagen morphology of control and tumor sample. The scale bar for control is 0.5 μm, while the scale bar for tumor is 2 μm. Vertical bars represent height in 80 nm. FIG. 5B plots the elastic modulus of lung control and tumor. Bars represent the means, and error bars are standard deviations. FIG. 5C plots the spectra of different spots of control and tumor tissue. FIG. 5D provides OPTIR images of CH and NH region of control and tumor tissue. Scale bars, 2 μm.

FIGS. 6A-6B illustrates representative VSFG spectra of control and tumor tissue, respectively. FIGS. 6C-6D show statistical results for NHS/CH_{2,Ss} and CH_{S}/CH_{2,Ss}, respectively, based on the spatially resolved VSFG spectra at control and tumor tissues. FIG. 6E provides VSFG images of CH_{2,Ss}, CHₛ, and NH_{S} mode of control and tumor tissue. The scale bar is 20 μm. Bars represent the means, and error bars are standard deviations.

FIGS. 7A-7F show VSFG peak ratio statistics and the corresponding hyperspectral images, where FIGS. 7A-7B provide the statistic results of NH_{S}/CH_{2,Ss} and CH_{S}/CH_{2,Ss}, respectively, of the mouse control and tumor tissue. The bars show the ratio distribution of the training area of control samples, the test area of control samples, and the image of tumor sample. Note the training and test data of the control samples yield similar distributions and therefore overlap with each other in this plot. FIG. 7C shows the NH_{S}/CH_{2,Ss} and CH_{S}/CH_{2,Ss} ratio mapping of the mouse control and tumor tissue. The scale bar is 20 μm. FIG. 7D plots spatially resolved VSFG spectra show the CH and NH region of tumor sample. FIG. 7E is a bright field image of tumor tissue after H&E stain. It is observed from the H&E staining image that the detected collagen is surrounded by tumor cells. The scale bar is 100 μm. FIG. 7F is an overlaid image of the VSFG image and the H&E image of tumor tissue, taken from the region within the dashed box in FIG. 7E.

FIGS. 8A-8C show results for human lung samples, where FIG. 8A provides VSFG images of CH_{2,Ss}, CHₛ, NH_{S} mode and SHG image of human control and tumor tissue. The scale bar is 15 μm; FIG. 8B plots statistic results NH_{S}/CH_{2,Ss} and CH_{S}/CH_{2,Ss} of the human control and tumor tissue. FIG. 8C shows NH_{S}/CH_{2,Ss} and CH_{S}/CH_{2,Ss} ratio mapping of the human control and tumor tissue.

FIGS. 9A-9C show theoretical modeling to understand the original of the VSFG peak intensity changes. The selection of collagen fibril diameters (10, 20, 30 and 40 nm) was guided by employing differential interference contrast (DIC) microscopy enhanced with extraordinary image shift (EIS) techniques. These studies have revealed that collagen fibrils can exist in diameters below 40 nm. The collagen fibril distance range (0-100 nm) was carefully chosen to align with the coherence length (Lc), calculated to be around 130 nm. FIG. 9A is a diagram showing orientation of vibrational modes relative to collagen fibril direction. FIGS. 9B and 9C plot the effect of collagen fibril distance and diameter on the ratios of CH_{S}/CH_{2,Ss} and NH_{S}/CH_{2,Ss} modes, respectively.

FIG. 10 displays a diagram of an exemplary experimental design according to an embodiment of the inventive scheme. The VSFG microscope is utilized to image and to analyze biological tissues containing collagen. Left: Schematic diagram of the VSFG microscope. Middle: Imaging of different vibrational modes of collagen in lung tissue. Right: Full VSFG spectra comparing tumor collagen with healthy collagen. Bottom: Direct imaging of tumor tissue based on the hyperspectral image of collagen from the VSFG microscope.

### DETAILED DESCRIPTION OF EMBODIMENTS

The disclosure provides a multimodal hyperspectral imaging platform to obtain broadband vibrational sum-frequency generation (VSFG) images, along with brightfield, second harmonic generation (SHG) imaging modalities. Due to the infrared frequency being resonant with molecular vibrations, microscopic structural and mesoscopic morphology of the material can be obtained of symmetry-allowed samples by the platform.

VSFG, a second-order nonlinear optical signal, has traditionally been used to study molecules at interfaces as a spectroscopy technique with a spatial resolution of ˜100 μm. However, the spectroscopy is not sensitive to the heterogeneity of a sample. Provided herein is a multimodal, rapid hyperspectral imaging platform of VSFG microscope. The spatial resolution of the VSFG spectroscopy platform reached the limit of ˜1 μm level, enabling the assessment of the structures of mesoscopically heterogeneous material samples. This imaging technique not only can resolve sample morphologies through imaging, but also record a broadband VSFG spectrum at every pixel of the images. Being a second-order nonlinear optical technique, its selection rule enables the visualization of non-centrosymmetric or chiral self-assembled structures commonly found in biology, materials science, and bioengineering, among others. As disclosed herein, an inverted transmission design allows for imaging unfixed samples. The disclosure provides that VSFG microscopy is capable to resolve chemical-specific geometric information of individual self-assembled sheets by combining it with a neural network function solver. Lastly, the images obtained under brightfield, SHG, and VSFG configurations of various samples provide the unique information revealed by VSFG imaging platform.

With advances in high numerical aperture (NA), reflective-based microscope objectives (Schwarzschild and Cassegrain geometries), which are nearly free of chromatic aberrations, the focus size of the two beams in VSFG experiments can be decreased from 100 μm² to 1-2 μm² and in some cases submicron. As disclosed herein, an inverted optical layout and broadband detection scheme were implemented, which enables a seamless collection of multimodal images (VSFG, SHG, and brightfield optical). The multi-modality imaging allows quick inspection of samples using optical imaging, correlating various types of images together, and locating signal positions on the sample images. With the achromatic illumination optics and choice of pulsed laser illumination source, this optical platform allows for future seamless integration of additional techniques such as Fluorescence microscopy and Raman microscopy, among others.

In one aspect, samples such as hierarchical organizations and a class of molecular self-assemblies (MSAs) were studied. These materials include collagen and biomimetics, where both the chemical composition and geometric organization are important to the ultimate function of the material. Because VSFG is a second-order nonlinear optical signal, it is specifically sensitive to intermolecular arrangements, such as intermolecular distance or twisting angles, making it an ideal tool for revealing both chemical compositions and molecular arrangements. The disclosure describes the VSFG, SHG, and brightfield modalities of the core instrument consisting of an ytterbium-doped cavity solid-state laser that pumps an OPA, a custom-built multimodal inverted microscope and monochromator frequency analyzer coupled to a two-dimensional CCD detector. A step-by-step construction and alignment procedures, and a complete part list of the setup, are provided herein. An in-depth analysis of exemplary materials demonstrates that the VSFG platform can reveal molecule-specific geometric details of organized matter. Additionally, it was shown that chemical-specific geometric details of the MSA can be determined using a neural network function solver approach.

As disclosed herein, the VSFG microscope uses a co-linear scheme, with a Schwarzschild objective serving as the condenser to achieve high spatial resolution (1.6 microns). It also employs a line-scanning technique to accelerate scanning speed. Additionally, it offers multimodal capabilities, including VSFG, SHG, and white-light imaging. Overall, it provides higher spatial resolution, faster scanning speeds, and multimodal functionality.

The construction of the VSFG microscope, the processes, data collection and data analysis are demonstrated in the following.

### Hyperspectral Line-Scanning VSFG Microscope

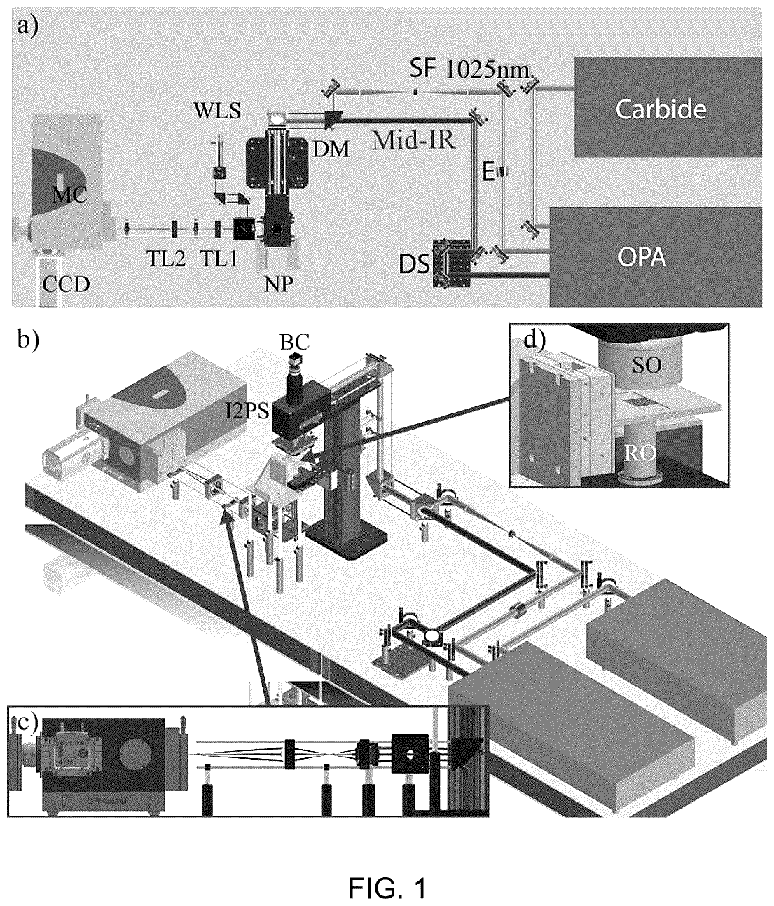

The 3D schematic of an exemplary VSFG microscope platform is shown in FIG. 1. Multiple components of the microscope include: MC, Monochromator; CCD, Charge-Coupled Device; WLS, White light source; NP: Nano positioner; DM, dichroic mirror; DS, Delay stage; RS, resonant scanner; RC, reflective objective; SO, Schwarzschild objective; RO, refractive objective; TL1, convex lens 1 in tube lens; and TL2, convex lens 2 in tube lens. The VSFG microscopy platform employs a pulsed laser system (Light ConversionCarbide). The laser beam preferably operates ˜100 fs time duration. In the exemplary set-up, the laser beam is centered at 1025 nm±5 nm (as shown in FIG. 1). The laser can operate at 40 W, 200 kHz (200 μJ/pulse) with a pulse width of ˜290 fs, as one example. The output of the seed laser was guided into an optical parametric amplifier (OPA, Light Conversion) to generate a mid-infrared (MIR) beam, and the frequency can be continuously tuned. As one example, the MIR is centered at 3450 nm±85 nm (˜2900±72 cm⁻¹), which encompasses part of the —CHₓ and —NH functional group region. As another example, thee MIR is centered at 6000 nm, probing the Amid groups, with a pulse duration of ˜290 fs and pulse energy of ˜6 μJ.

For the up-conversion near IR (NIR) beam, the residual laser beam from OPA was passed through a Fabry-Perot etalon to produce a spectrally narrowed up-conversion beam with an FWHM of ˜4.75 cm⁻¹. The polarization of the beam pulse and MIR beam were controlled with their corresponding λ/2 waveplates.

Both up-conversion NIR beam and MIR beam were spatially overlapped at a customized dichroic mirror (DM in FIG. 1) that is transmissive to MIR and reflective to NIR. The overlapped beams were directed into an inverted microscope with an integrated 325 Hz single-axis resonant beam scanner (Mounted to integrated two-position scanner (I2PS) in FIG. 1). The resonant scanner projected a line of the two overlapped beams onto the sample through the condenser objective. The resonant scanner was mounted to a slider which enabled the seamless reconfiguration between VSFG/SHG and brightfield modalities. The two spatially overlapped beams were focused onto the sample with a purely reflective Schwarzschild objective (0.70 NA, PIKE Technologies Inc., PN 891-0007) as shown in FIG. 1. The VSFG signal generated by the sample was collected with an infinity, refractive microscope objective (Zeiss, Fluar 0.75 NA, working distance=0.6 mm) (FIG. 1), which was guided through a linear polarizer and then through a telecentric tube lens system composed of two f=60 mm focal lenses (TL1 and TL2 in FIG. 1). The magnified image from the tube lenses was formed at the entrance slit of the monochromator and the spatially/frequency resolved data was detected on a two-dimensional CCD detector (CCD in FIG. 1). To switch to SHG imaging, one can block the IR beam and rotate the grating of the spectrograph to 501.5 nm to image the SHG signal.

The details of the operation protocols are the following.

### Laser System

Use a pulsed laser system, centered at 1025 nm±5 nm, as an example. The laser is set at 40 W, 200 kHz (200 μJ/pulse) with a pulse width of ˜290 fs, in the exemplary set up. The exact repetition rate can vary, and a high repetition rate laser generally works better for this VSFG microscope.
Guide the output of the seed laser into an optical parametric amplifier (OPA) to generate a mid-infrared (MIR) beam. Tune the MIR to the frequency of interests (FIG. 1).

### Up-Conversion Beam

Pass the residual beam from OPA through a Fabry-Perot etalon to produce a spectrally narrowed up-conversion beam with an FWHM of ˜4.75 cm⁻¹.
Spatially filter the narrowed 1025 nm beam with an 8 μm sapphire pinhole. The 1025 nm beam can be visualized using a NIR card. And control the polarization of the 1025 nm pulse with a λ/2 waveplate.

### MIR Beam

Guide the MIR beam through a delay stage for fine control of the temporal overlap.
Control the polarization of the MIR with a λ/2 waveplate.

### VSFG Microscope

Spatially overlap both up-conversion NIR, and MIR beams at a customized dichroic mirror (DM, FIG. 1b) that is transmissive to MIR and reflective to NIR. Use two irises to guide the alignment: one right after the DM, and one at the far end. Use a power meter after the iris to determine whether MIR is centered, and use a NIR card to locate NIR positions.
After the overlap, the NIR beam can be used to guide both beams.
Direct the overlapped beams into an inverted microscope with an integrated 325 Hz single-axis resonant beam scanner (mounted to an integrated two-position scanner (I2PS), FIG. 1, Panel b.
The resonant scanner projects a line of the two overlapped beams onto the sample through the condenser objective. It is mounted to a slider which enables the seamless reconfiguration between VSFG/SHG and brightfield modalities.
Focus the two spatially overlapped beams onto the sample with a purely reflective Schwarzschild objective (SO, FIG. 1, Panels b,d).
Collect the VSFG signal generated by the sample with an infinity-corrected refractive objective (RO, FIG. 1, Panels b,d).
Guide the collimated output VSFG signal through a linear polarizer and then through a telecentric tube lens system composed of two f=60 mm focal lenses (TL1 and TL2, FIG. 1, Panels b,c).
The magnified image from the tube lenses is formed at the entrance slit of the monochromator (MC, FIG. 1, Panels b,c), and the spatially/frequency resolved data is detected on a two-dimensional CCD detector (CCD, FIG. 1, Panel b).

### SHG Mode

To switch to SHG imaging, block the IR beam and rotate the grating of the spectrograph to the second harmonic of the upconversion pulse, for example 501.5 nm, to image the SHG signal.

### Brightfield Mode

To switch to brightfield optical imaging, turn on the white light source, and move the integrated slider (I2PS, FIG. 1, Panel b) to collect brightfield images in the counter-propagating direction, with the imaging objective (RO) acting as the condenser and the condenser objective (SO) acting as the imaging objective.

Form an image of the collimated output of the refractive objective at the sensor plane of an RGB brightfield camera using a commercially available tube-lens system.

### Hyperspectral Microscope Alignment and Vertical CCD Axis Spatial Calibration

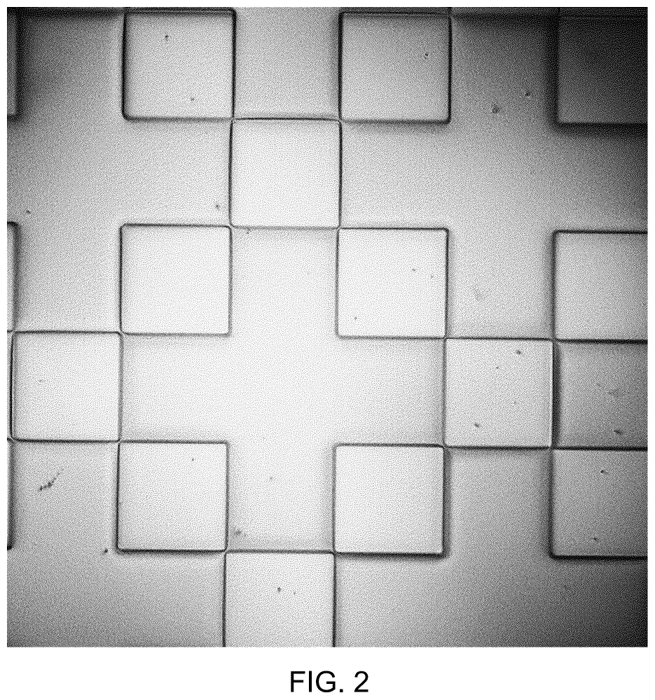

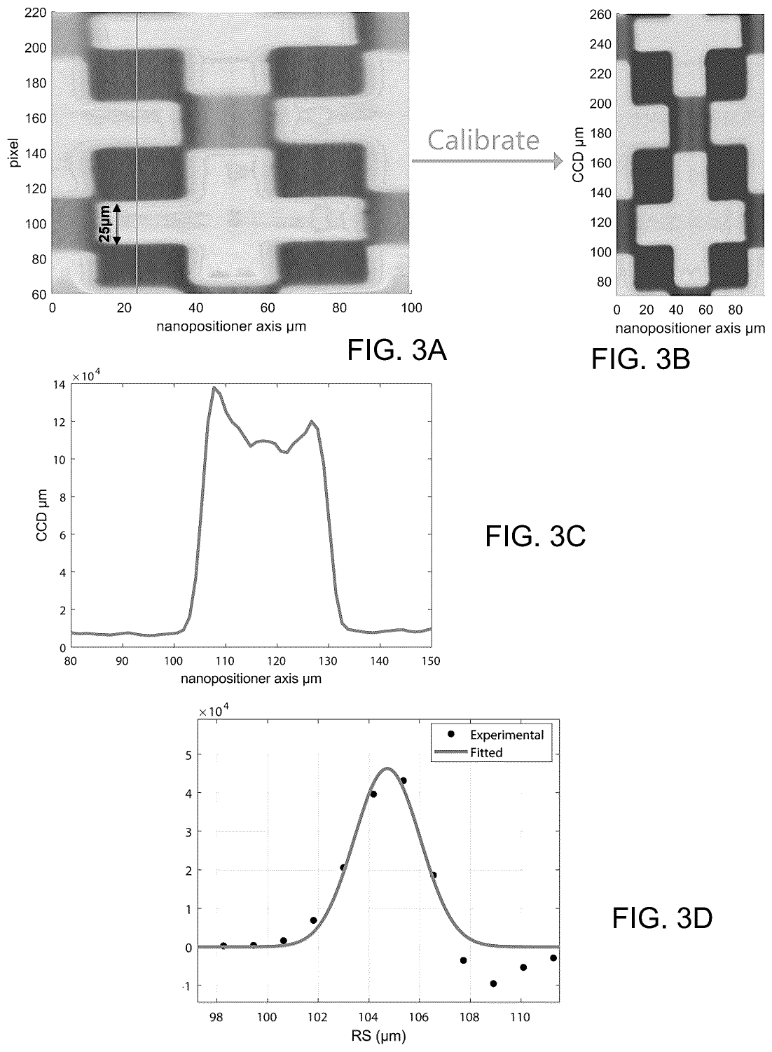

Roughly optimize the position of the sample plane (nano positioner z-axis) using a standard sample of ZnO (1 μm thick) pattern sputter coated 15 mm×15 mm×0.170 mm±0.005 mm coverslip and bringing it into brightfield focus using the brightfield imaging modality.
The z-position of the RO as well as the alignment of the white light, may need to be adjusted as necessary. A representative image of the ZnO on the glass pattern used for alignment calibration is shown in FIG. 2.
Move the I2PS back to the nonlinear illumination arm and optimize the sample height for the maximum nonresonant VSFG intensity generated by the ZnO regions observed on the CCD camera.
The z-position of the RO must be adjusted for maximum intensity. One may have to iterate the previous steps a few times before the optimal height of the sample, and RO are reached.
Turn the resonant beam scanner on and collect a line of the images.
Collect nonresonant intensity images by scanning the sample perpendicularly to the beam scanner direction. Take vertical slices of the image data and establish the pixel: micron ratio. (shown in FIGS. 3A, 3B).
The derivative of these line sections is analyzed to produce the vertical CCD axis pixel: micron ratio that will be used for future images.

### Hyperspectral Data Collection.

Collect the spectra of a vertical line of the VSFG signals on the CCD, whose spectra are dispersed along the horizontal axis and spatial positions are recorded on the vertical axis of the CCD.
This results in a two-dimensional data set for a single-line section.
After the line section of the sample is hyperspectrally imaged, scan the sample in the axis perpendicular to the line scanning axis using the three-dimensional nano-positioner (NP, FIG. 1).
The 3D nano positioner is important for high precision and reproducibility in locating sample regions (x-y plane) as well as bringing the sample into focus (z-axis).
Iterate between the two steps to collect a VSFG hyperspectral image.

### Hyperspectral Data Analysis

Spectrally unmix the data using the MatLab® imaging toolbox hyperspectral imaging library workflow. NOTE: Spectral unmixing correlates spatial locations to unique spectra. MatLab® code for hyperspectral data analysis is provided in Table 1.
Create a 4-dimensional hypercube (x=spatial, y=spatial, z=frequency-dependent intensity, ω=frequency) using the hypercube function in the MatLab® image processing toolbox hyperspectral imaging library.
Identify the number of unique spectra with the countEndmembersHFC function with a probability of false alarm (PFA) value of 10-7.
Identify unique spectra using the nfindr spectral unmixing function.
Finally, using the sid function, associate each pixel with one of the unique spectra identified in the previous step.
Additional spectral unmixing and matching methods can be done with alternative functions/algorithms offered in the MatLab Hyperspectral Imaging Library.
Fit the sum data for each isolated sheet to the Voigt function.
Lorentzian function represents the pure homogeneous lineshape limit, whereas the Gaussian function originates from inhomogeneous limits. In reality, the systems could be in a combination of homogeneous and inhomogeneous limits, which requires a Voigt function—a common practice for condensed phase spectroscopy, including VSFG.

TABLE 1
MatLab ® Code for Hyperspectral Data Analysis

```
    data=load (′filename.ccdsfg′);
    data(:,1)=[ ];
    w=load (′w.txt′);% wavenumber
    y=256;% pixel number
    x=length(data(:,1))/y;% scan points along x axis
    z=length (w);
    i=1;
    b=zeros(y,x,z);
    while i<=z;
b(:,:,i)=reshape(data(:,i),[y,x]);
i=i+1;
    end,
    hcube = hypercube(b, w);%Create a 4-dimensional hypercube
    numEndmembers = countEndmembersHFC(hcube, ′PFA′,10\hat{ }−7);
    unmixedSpectra = nfindr(hcube, numEndmembers);
    figure;
    plot (w,unmixedSpectra);
  score = zeros(size(hcube.DataCube,1),size(hcube.DataCube,2),numEndmembers);
    for i= 1:numEndmembers
score(:,:,i) = sid(hcube,unmixedSpectra(:,i));
    end
    [~,matchingIndx] = min(score,[ ],3);
    figure;
    imagesc(flipud(matchingIndx))
```

### Application in Tumor Diagnosis

In one aspect, the VSFG microscope platform was applied for tumor diagnosis, employing tumor related collagen as a biomarker. Collagens, the most abundant proteins in the tumor microenvironment (TME), have been proposed as a biomarker for several types of cancers. For example, Type I collagen metabolites as tumor markers have been studied in patients with lung carcinoma (Cancer: Interdisciplinary International Journal of the American Cancer Society, 85(9), pp. 1951-1957). The large-scale tumor-associated collagen signatures can identify high-risk breast cancer patients (Theranostics 11, no. 7 (2021): 3229). Type IV collagen can be used as a tumor marker for colorectal liver metastases (European Journal of Surgical Oncology (EJSO), 37(7), pp. 611-617). Serum cross-linked carboxyterminal telopeptide of type I collagen (ICTP) is used as a prognostic tumor marker in patients with esophageal squamous cell carcinoma (Cancer, 94(4), pp. 940-949). Immunohistochemical study of type I collagen and type I pN-collagen in benign and malignant ovarian neoplasms is reported (Cancer, 75(4), pp. 1010-1017).

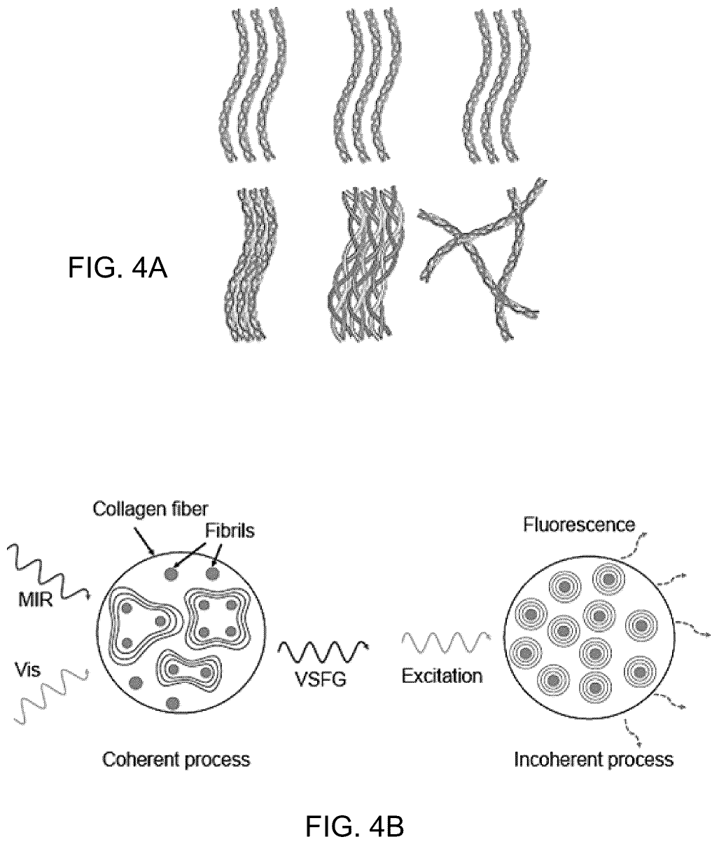

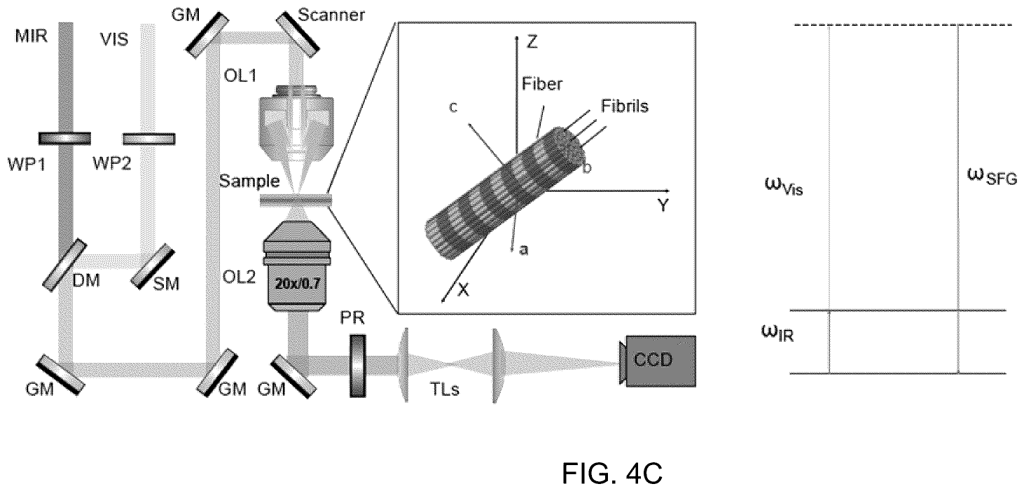

Collagens are major structural components of the extracellular matrix (ECM), and it is composed of a triple helix of polypeptide chains that further pack into collagen fibrils and assemble into fibers. A higher collagen content is related to higher mammographic density and predicts an increased risk of breast cancer in women. Pathological conditions such as fibrosis involve accumulations of collagens, leading to variations in fibril density, packing configurations, and changes in fiber orientation and crimp, which may exert profound effects on tumor progression (FIG. 4A). Different types of collagens in the stroma play either tumor-restrictive or tumor-permissive roles. Tumor formation and progression often result in alterations in collagen type, density, and architecture. In turn, the altered collagen network affects a variety of cellular behaviors in the tumor milieu to promote tumor progression. Using Atomic Force Microscopy (AFM), it has been shown that the mechanical properties of collagens, i.e. stiffness, in tumor ECM are much higher than in healthy tissues. Considering the reported changes in mechanical properties, it is natural to infer that the associated structural changes at microscopic or mesoscopic levels are detectable by molecular spectroscopy—the primary method for monitoring molecular transformations. More importantly, molecular spectroscopy can be developed into hyperspectral imaging for label-free tumor detection.

Label-free identification of tumor-related collagens by hyperspectral imaging is provided in the disclosure. Second harmonic generation (SHG) microscopy is a powerful modality for imaging fibrillar collagen in a diverse range of tissues. SHG is a 2^{nd} order nonlinear optical process generated from non-centrosymmetric systems, making it highly sensitive to the collagen fibril/fiber structure. This sensitivity arises from the coherent emission of SHG signal, which interfere with each other, making the signal sensitive to the distance, relative orientation and other alignments of the SHG emitter, i.e. collagen fibrils/fibers. In contrast, many other biological species lacking specific symmetry become transparent to SHG, rendering SHG highly sensitive to detecting collagens. However, it is difficult to infer collagen structures based on overall SHG intensity and to distinguish between collagens in healthy and tumor tissues, because SHG does not provide bond specific information.

On the other hand, various Raman microscopy techniques (stimulated Raman scattering and coherent anti-Stokes Raman scattering) and IR imaging have yet to be applied to directly differentiate tumor domain by collagen-based imaging, despite their success in pre-clinical animal models. One reason could be although they offer molecular sensitivity by revealing chemical composition changes of biomarker molecules through spectral peak shifts, not all tumor growth leads to substantial chemical changes detectable by vibrational peak shifts. Even if such changes occur, IR and Raman detect all molecules in the sample path, making these changes subtle due to significant spectral background from other intact biological and chemical species. More importantly, mesoscopic structure changes in collagen fibers cannot be detected by these techniques due to their lack of coherent interference effects.

The inventive approach combines the best of two worlds, namely, vibrational spectroscopy and second order nonlinear optics. The inventive VSFG microscopy platform provides a label-free imaging technique for tumor identification, leveraging its spectral sensitivity to fibril packing. VSFG platform employs two incident optical beams, one IR beam at ω_{IR} and another up-conversion beam at ω_{VIS}, to generate a signal at the sum frequency ω_{SFG}=ω_{IR}+ω_{VIS}. Because the IR beam resonates with molecular vibrational modes (as shown in FIG. 4B), it is sensitive to specific molecular modes, similar to IR or Raman spectroscopy. Furthermore, like SHG, VSFG is a 2^{nd} order coherent nonlinear optical process sensitive to non-centrosymmetric structures due to symmetry and coherent interferences. Within the same collagen fibers, vibrational modes originating from different chemical bonds interfere distinctively, depending on their relative orientation and arrangements in the collagen framework, similar to the pioneering works on VSFG imaging studies of cellulose fibers. In the disclosure, it is demonstrated that VSFG's unique advantage of combining structural selectivity with chemical-bond sensitivity makes its spectra exhibit sensitivity towards fibrils packing. As a result, the VSFG platform can differentiate collagen in complex biological environments without signal contributions from other biological species.

In one embodiment, hyperspectral images of collagens were collected by a fast line-scanning VSFG microscopy with 1.6 micron spatial resolution. Within each pixel of the image, a broadband VSFG spectrum encoding the collagen structural information was recorded. Two spectral signatures were identified—the intensity ratio of NH_{S}/CH_{2,Ss} and the ratio of CH_{S}/CH_{2,Ss} modes—to discern lung tissues bearing metastatic tumors and tumor-free lung tissues. These signatures were implemented to differentiate tumor from normal tissue in both mouse and human lung samples. Based on the DFT calculation and the VSFG theory, this contrast is explained by demonstrating that different spectral peaks have distinctive sensitivity to interfibrils spacings. The spectral observation suggests denser collagen packing in tumors, corroborating the enhanced stiffness of the tissues. Thus, the disclosed techniques demonstrate the power of VSFG in identifying collagen fibers in tumor tissues, providing a new diagnostic modality for oncology. Furthermore, the structural knowledge at the molecular scale gained by hyperspectral imaging offers new insights for fundamental biophysics research, and pathology.

As disclosed herein, VSFG microscopy platform was provided as a new label-free imaging technique for tumor identification, combining the advantages of chemical-bond selectivity from vibrational spectroscopy and coherent interference from second-order coherent nonlinear optics. Using the fast line-scanning VSFG microscope, the hyperspectral VSFG images of collagen I were obtained from both lung tissues bearing metastatic tumors and tumor-free ones, which show drastic different spectral signatures: tumor samples exhibit large NH stretch (NH_{S}) and CH stretch (CH_{S}) versus the CH₂ symmetric stretch (CH_{2,Ss}), in comparison with the healthy controls. Two spectral signatures were identified—the intensity ratio of NH_{S}/CH_{2,Ss} and the ratio of CH_{S}/CH_{2,Ss} modes—to distinguish tissues from metastatic tumors from one of tumor-free. These signatures demonstrated high fidelity in distinguishing tumors from normal tissue in both mouse and human lung samples. Theoretical modeling further explained this contrast by demonstrating that different spectral peaks interfere distinctively when varying the interfibrilar distances—resulting dramatic peak ratio changes. The disclosure demonstrated that collagen fibrils pack denser in tumors, corroborating the enhanced stiffness of the tissues. VSFG microscopy, as a new label-free method, could contribute to high sensitivity, real-time characterization while preserving sample integrity in oncology, pathology and fundamental biophysical researchers.

### Experimental Methods

### Mechanical and IR Imaging Studies of Mouse Lung Tissues

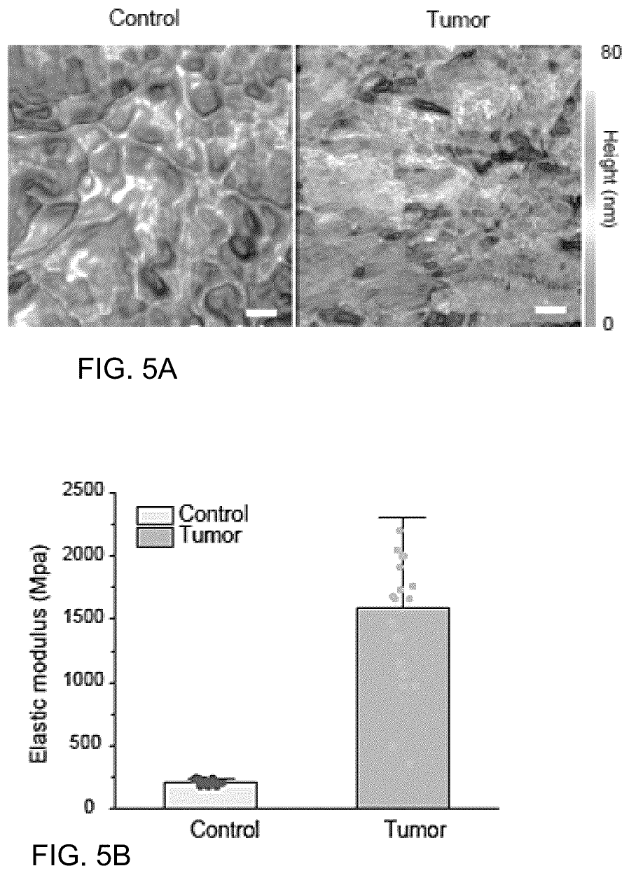

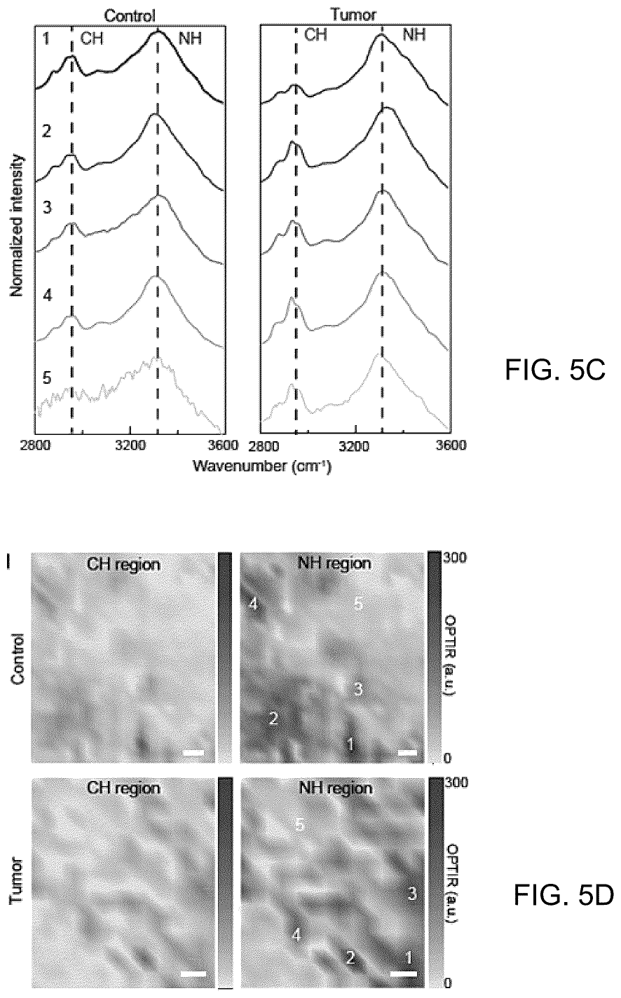

The stiffness between normal tumor-free mouse lung tissues and those bearing metastatic tumors was compared. The metastatic lung tumors were developed following tail-vein injection of MDA-MB-231 human breast cancer cells into female NOD-scid IL2Rgamma^{null} mice. Using AFM, it was found that tumor and control collagen fibers exhibited similar topographic maps (FIG. 5A). However, force maps of the respective regions revealed differences in the elastic properties of the collagen-rich ECM in tumor tissues compared to control tissues. The measured elastic modulus of collagen in the control sample was ˜200 MPa (FIG. 5B), consistent with previous reported value. In contrast, the elastic modulus for tumor tissues was 1500 MPa. This significant difference in elastic modulus indicated that collagen rich sites in tumors were up to 7.5-fold stiffer than the controls.

The changes in stiffness could originate from microscopic structural changes, such as modifications of amino acid or peptide chemical structures, or mesoscopic alternations, such as fibril/fiber packing and alignment. To examine the former, an optical photothermal infrared (OPTIR) microscopy was employed. OPTIR microscopy measures infrared absorption spectra at each site through photothermal expansion and scans the samples to attain hyperspectral images. For both healthy and tumor tissues, their spectral profiles exhibited striking similarities, characterized by pronounced CH vibrational modes (2800-3000 cm⁻¹) and a broadband peak spanning the NH (3200-3500 cm⁻¹) regions (FIG. 5C). As a result, the intensity map of CH and NH vibrational modes for both tumor and control samples resembled each other (FIG. 5D). Furthermore, it is worth mentioning that all pixels in the image showed IR signal even when it appears weak (e.g., spot 5 in FIG. 5C). This is because all molecules (e.g. proteins, lipids.) in the tissue contributed to IR absorption, as long as they possess the corresponding chemical bonds. This uniformity in the spectral characteristics and identical spatial distribution of CH and NH modes suggested that there were no dramatic chemical differences between healthy and tumor tissues, such as bond formation or breaking. This result also confirms that IR imaging lacks the power to differentiate tumor from healthy tissues based on chemical composition alone.

### VSFG Spectra Analysis and Tumor Identification Based on Hyperspectral Imaging of Mouse Lung Tissues

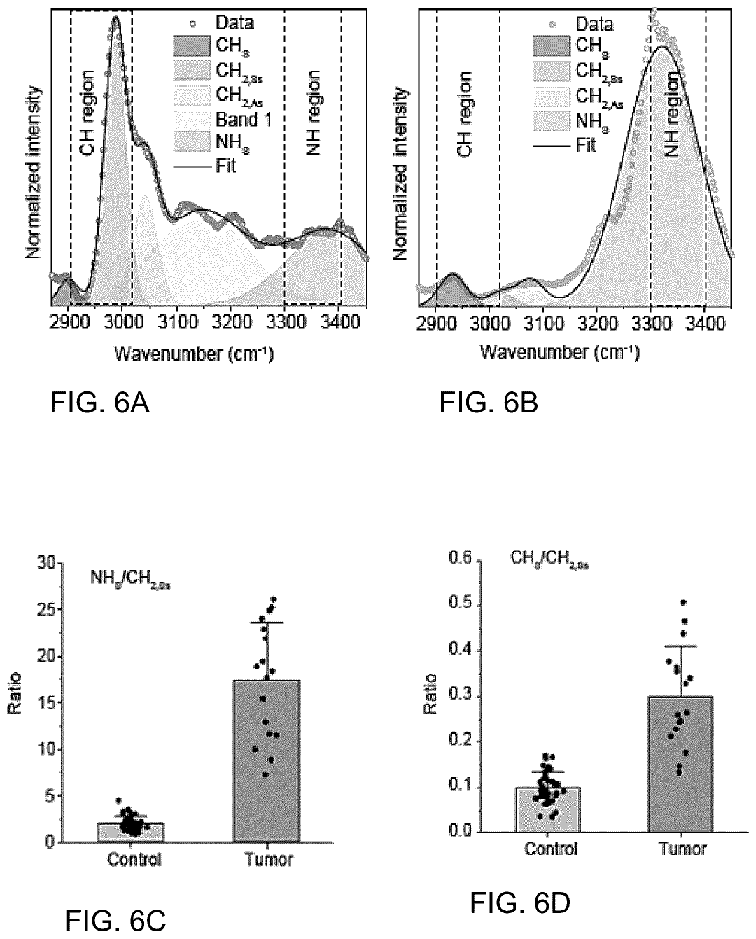

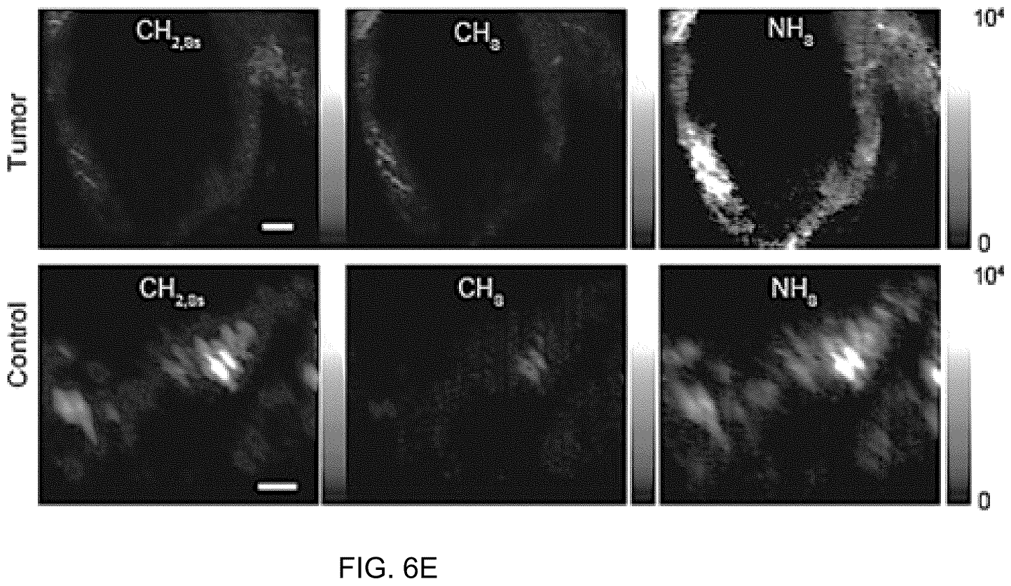

Because OPTIR results showed dominant vibrational signals of the CH and NH modes, the spatially resolved VSFG spectra in these spectral regions were measured. The VSFG spectra of control and tumor samples exhibited substantial differences (FIGS. 6A-6B). The VSFG spectra of the healthy sample showed two major peaks near 2970 and 3050 cm⁻¹ respectively, on top of a broad feature, whereas the tumor tissues displayed a large peak near 3350 cm⁻¹. Gaussian functions were used to fit these bands. For the control sample, the dominating peaks located at 2970 cm⁻¹, 3050 cm⁻¹ and 3310 cm⁻¹ were assigned to CH₂ symmetric stretch (CH_{2, Ss}), CH₂ asymmetric stretch (CH_{2,As}) and amide A band (associated with the NH-stretch, NH_{S}), respectively, according to literature and our calculation. A side peak at around 2905 cm⁻¹ is assigned to CH stretch and a broadband peak at 3150 cm⁻¹ might result from the amide B or non-resonant signal that is not the focus of this study.

In contrast, for the tumor sample, the relative CH_{2,Ss} was suppressed, while CH_{S} became apparent and NH_{S} dominated (FIG. 6B). It was noted the NH_{S} peak in tumor sample red-shifted to 3330 cm⁻¹. The NH_{S} peak is reported to be located at 3200-3400 cm⁻¹ in literature⁵⁵, and the peak would redshift as the NH of the peptide group participates or experiences a strengthening in hydrogen bonds. Noticeably, to eliminate the influence of water vapor absorption on the NH speak region, all the experiments were performed under dry air purging condition. It was concluded that OH stretch have a negligible effect on the studied spectra region (2800-3400 cm⁻¹).

Overall, the VSFG spectra in FIGS. 6A-6B suggested that the relative intensity of NH_{S} signal was much higher than the CH_{2,Ss} in the tumor sample, while these two peaks had comparable intensities in the healthy sample. Thus, the peak intensity ratio between NH_{S} and CH_{2,Ss} stretch could be used as a spectral signature to identify tumor tissues. To determine if this signature was statistically significant, we randomly measured VSFG spectra at 44 different spots in control samples and 17 spots in tumor samples. The NH_{S}/CH₂. Ss ratio for control sample is 2.0±0.7, while the NH_{S}/CH_{2,Ss} ratio for tumor sample is 17±6 (FIG. 6C). These results confirm that statistically, the NH_{S}/CH_{2,Ss} ratio is dramatically different between these two samples.

The hyperspectral image was performed at CH_{2, Ss} (IR centered at 2941 cm⁻¹) and NH_{S} (3333 cm⁻¹) region (FIG. 6E), by setting our IR laser at these specific frequencies, to explore the capability of differentiating tumor tissues based on VSFG image using specific vibrational modes. Unlike OPTIR, the VSFG images only show strong intensities in specific regions, because VSFG is sensitive to non-centrosymmetric collagen structures, while other centrosymmetric TMEs did not appear. This selectivity eliminated the unwanted background signal and allowed us to follow changes in collagen tissue signals. Most importantly, the VSFG images showed drastic differences between the tumor and control tissues. For control sample, VSFG images revealed that the overall CH_{2,Ss} and NH_{S} had similar intensity and spatial mapping (note all images were displaced using the bar for comparison, FIG. 6E, bottom). Conversely, the tumor sample presented a much higher NH_{S} mapping intensity than CH_{2,Ss} (FIG. 6E, top). The observed VSFG image intensity difference was consistent with the one from VSFG spectra (FIGS. 6A-6B).

### Data Analysis

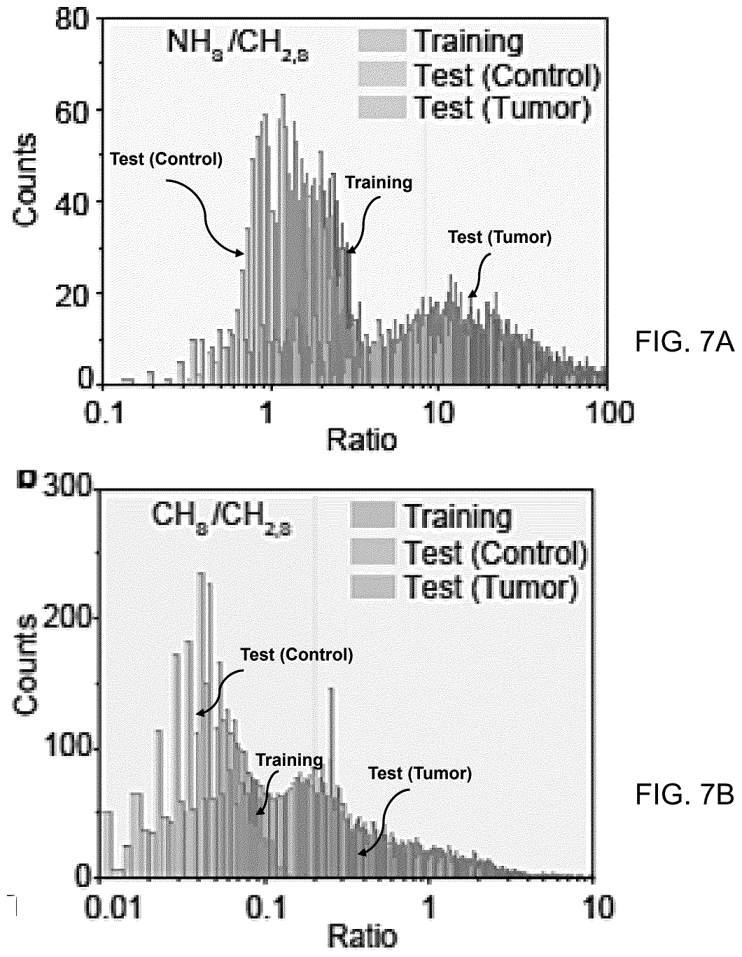

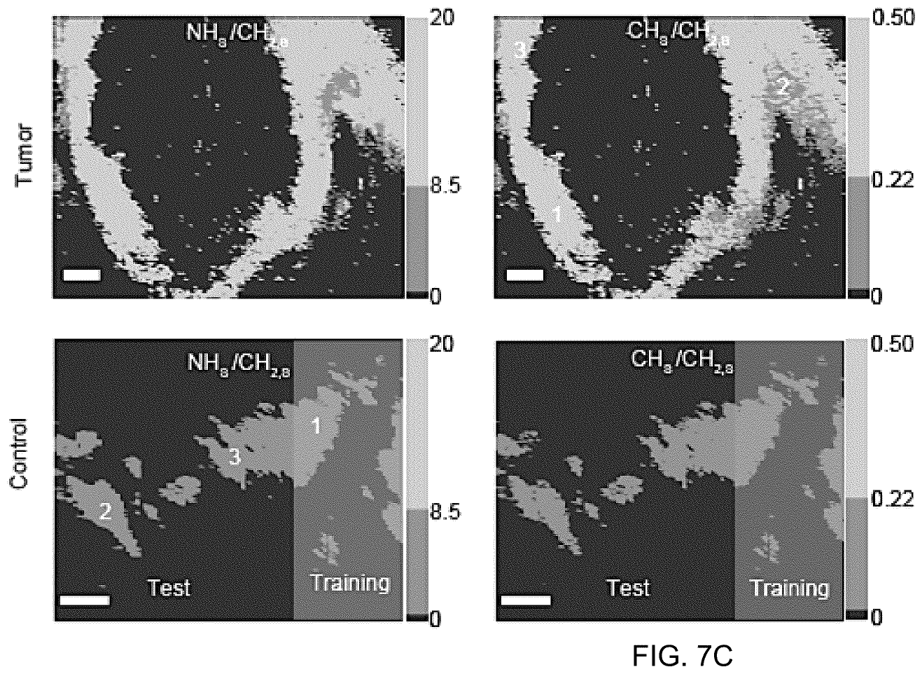

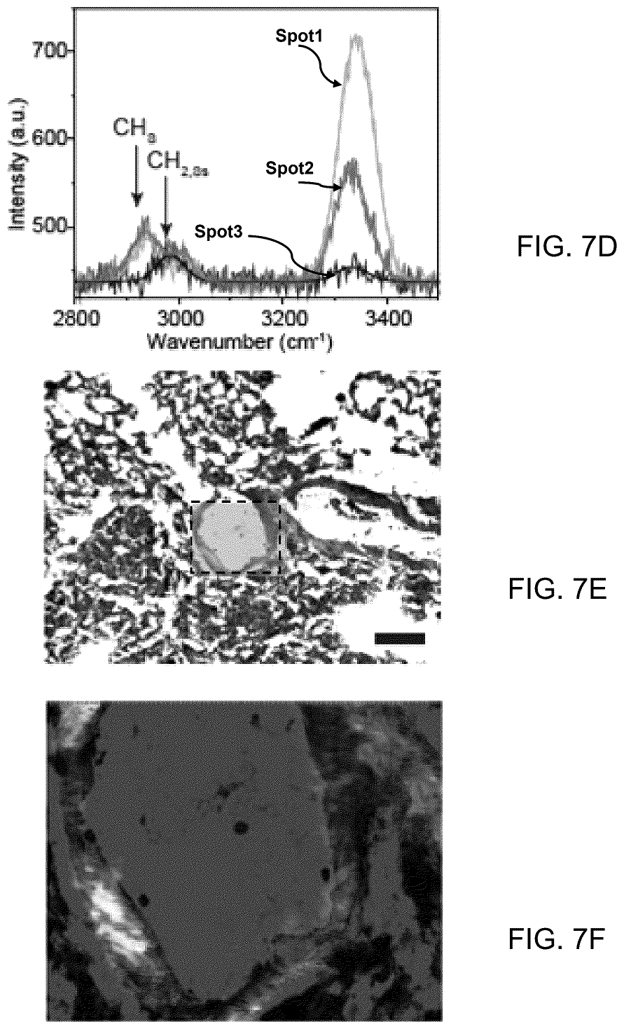

The calculation was made on the image intensity ratio from an area of the control sample image, used as the training data sets (highlighted in FIG. 7C). It was found that the control sample showed an average NH_{S}/CH_{2,Ss} ratio 2.5, ranging from the 10th to the 90th percentile of 1.5 to 3.4. In contrast, the average NH_{S}/CH_{2,Ss} ratio obtained from the entire tumor sample image is much higher ˜56, with a range from the 10th to the 90th percentile of 6.3 to 127 (Table 2). The substantial ratio differences indicate that a threshold can be determined based on the image intensity ratio of NH_{S}/CH_{2,Ss} to directly detect the tumor domain in the hyperspectral images.

From the NH_{S}/CH_{2,Ss} ratio distribution histogram (FIG. 7A), the control sample exhibited a Gaussian-like distribution, while the tumor sample showed some overlaps with the control one but extended to a much large numerical region. This overlap could be explained by the presence of healthy domains within the tumor sample. This is reasonable because even in tumor samples, not all the collagen tissues need to be fully degraded. Based on the histogram, it was decided to use the upper bound 99.7% confidence interval of the NH_{S}/CH_{2,Ss} ratio of control samples (i.e. the mean plus 3 times of the standard deviation) as the threshold to differentiate tumor and control domains, leading to a boundary ratio of 8.5. This method ensured that nearly all testing area of control samples were below the threshold and maintained high sensitivity for detecting the tumor domain.

Using this threshold, tumor samples demonstrated a spatially dependent NH/CH_{2,Ss} ratio, with most areas above the threshold (FIG. 7C). In contrast, for control (healthy) samples, the NH_{S}/CH_{2,Ss} ratio was consistently well below the threshold (FIG. 7C). Remarkably, the area showing large NH_{S}/CH_{2,Ss} overlaps with the tumor tissue area identified by the hematoxylin and eosin (H&E) staining methods (FIGS. 7E-7F), validating that the NH_{S}/CH_{2,Ss} ratio is a reliable spectral identifier for tumor tissue. (Note: the samples measured by VSFG microscopy and the stained samples are two consecutive slices from the same tissue sample, so their tumor areas are directly related). Overall, the drastic contrast of NH_{S}/CH_{2,Ss} ratio between the healthy and tumor tissues can serve as a robust signature in tumor identification.

There is one practical inconvenience in using the NH/CH_{2,Ss} ratio. In many VSFG systems, the two spectral regions are so far apart that it requires scanning the IR wavelength, which lengthens the imaging process. Thus, it is desirable to find signatures with peak frequencies close to each other, so that it is possible to obtain the hyperspectral VSFG images without laser frequency scanning. It was noticeable that as the NH_{S} dramatically increases its intensity in tumor samples, the CH_{S} stretch signal also becomes stronger relative to the CH_{2,Ss} stretch, despite the overall spectral feature is weak (FIGS. 6A-6B). This observation was further confirmed when plotting the CH_{S}/CH_{2,Ss} ratio extracted the VSFG spectra; tumor samples exhibit large CH_{S}/CH_{2,Ss} ratio, statistically, whereas the control samples display negligible ratios (FIG. 6D).

Following the same treatment of the NH/CH_{2,Ss} ratio, the statistics of CH_{S}/CH_{2,Ss} ratio of the training image (shown in FIG. 7B) exhibit a value of 0.07 for control sample, with a range from the 10th to the 90th percentile of 0.04 to 0.11. For the tumor sample, the ratio is 1.13, with a range from the 10th to the 90th percentile of 0.11 to 2.52 (Table 2). It is shown that the CH_{S}/CH_{2, Ss} intensity ratio is 10 times larger for the tumor samples than the healthy control, and a threshold of 0.22 (the upper bound of 99.7% confidence level of the control sample) is extracted, to differentiate tumor from healthy tissues.

When applying this threshold value of 0.22 for the CH/CH_{2 Ss} ratio to differentiate tumor and healthy tissues, we found all test area of healthy tissue showed ratio smaller than the threshold, while the tumor tissues showed a large domain above the threshold (shown in FIG. 7C) and a few small domains below it. The above threshold areas with CH_{S}/CH_{2,Ss} ratio largely coincided with those identified by NH_{S}/CH_{2,Ss} ratio, suggesting that CH_{S}/CH_{2,Ss} had a similar ability to identify collagen related to tumor tissues. It was also interesting to notice the good agreement between the areas displacing the NH_{S}/CH_{2,Ss} and CH/CH_{2,Ss} ratios below their corresponding thresholds (shown in FIG. 7C). Indeed, by inspecting spectral of a few specific spots, the spots that displayed a high NH_{S} peak always had a visible CH_{S} feature (FIG. 7D), which was absent in control sample. This consistency in the spatial mapping further indicated that the collagen fibers of a small region in the tumor tissue maintain their normal structures.

### Tumor Diagnostics

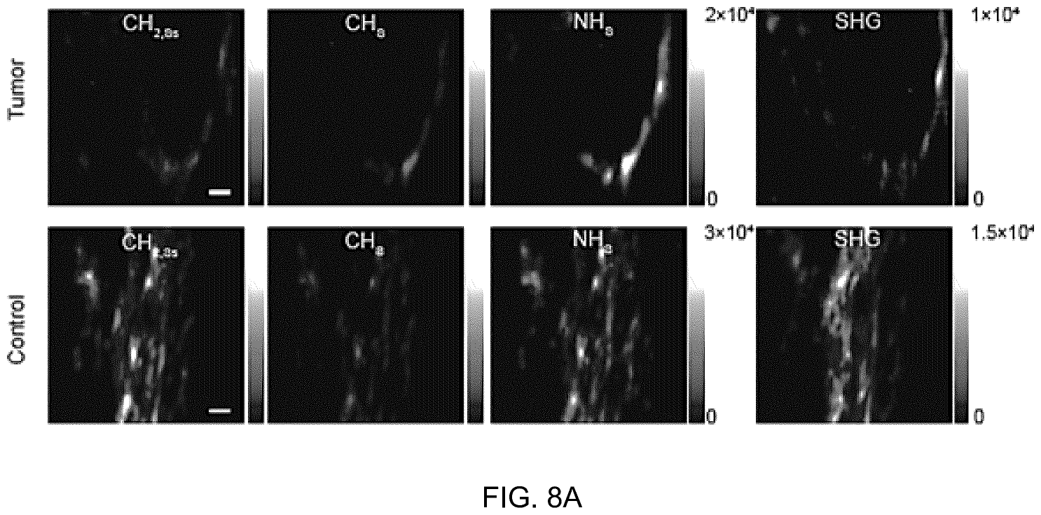

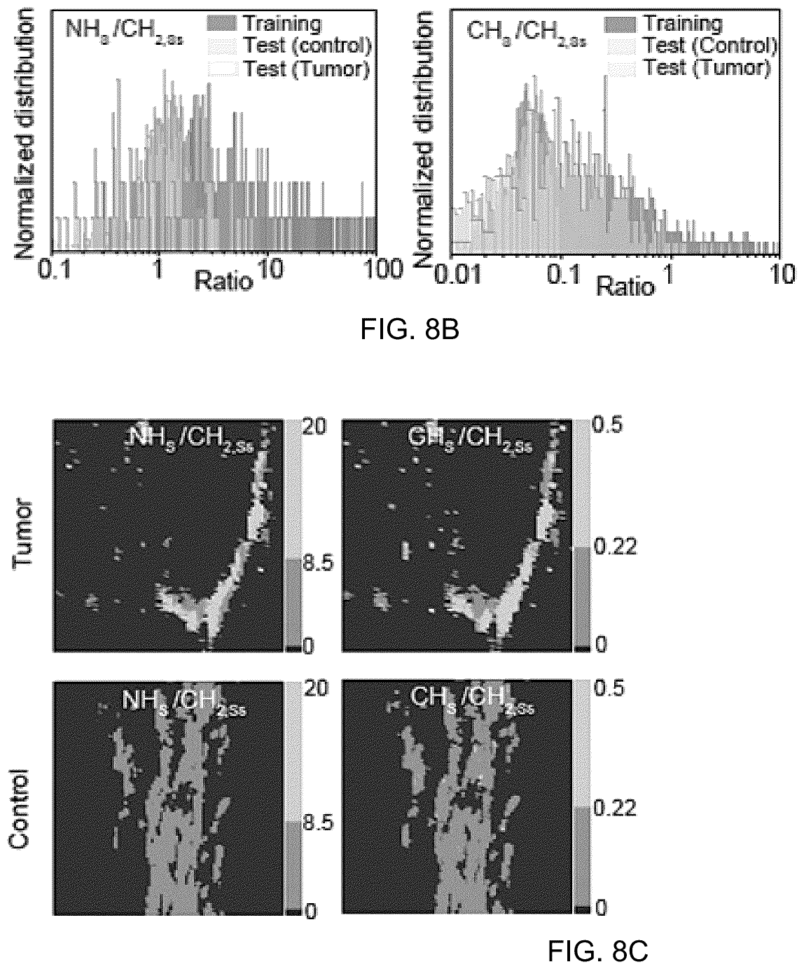

The results of mouse lung tissues indicated VSFG microscopy could directly image tumor domain based on the NH_{S}/CH_{2,Ss} and CH_{S}/CH_{2,Ss} ratio mapping. To examine whether this observation can be extended to tissues of other mammals, hyperspectral imaging on human lung tissues was performed. The hyperspectral VSFG images of CH_{2,Ss}, CH_{S}, and NH_{S} vibrational mode in FIG. 8A showed that the NH_{S} signal was much stronger than that of CH_{2,Ss} in tumor, whereas they were comparable in control samples—similar to the mouse model.

Based on the statistical analysis shown in FIG. 8B, both the NH_{S}/CH_{2,Ss} and CH_{S}/CH_{2,Ss} ratio in tumor tissues exhibited substantial differences compared to control samples. As summarized in Table 2, the mean values, 10th and 90th percentile of both NH_{S}/CH_{2,Ss} and CH_{S}/CH_{2,Ss} in the tumor sample are much higher compared to the control sample. These substantial differences indicate that we could use also the NH_{S}/CH_{2,Ss} and CH_{S}/CH_{2,Ss} ratio to differentiate the human tumor tissue from the healthy ones.

TABLE 2
Statistic Results of Mouse and Human Lung Samples

| Sample | | Ratio | Mean | 10 th | 90 th |
|---|---|---|---|---|---|
| Mouse | Control | NH_{S}/CH_{2, Ss} | 2.5 | 1.5 | 3.4 |
| | | CH_{S}/CH_{2, Ss} | 0.07 | 0.04 | 0.11 |
| | Tumor | NH_{S}/CH_{2, Ss} | 56 | 6.3 | 127 |
| | | CH_{S}/CH_{2, Ss} | 1.13 | 0.11 | 2.52 |
| Human | Control | NH_{S}/CH_{2, Ss} | 1.9 | 0.8 | 3.5 |
| | | CH_{S}/CH_{2, Ss} | 0.08 | 0.02 | 0.16 |
| | Tumor | NH_{S}/CH_{2, Ss} | 20 | 1.2 | 44 |
| | | CH_{S}/CH_{2, Ss} | 0.88 | 0.09 | 2.3 |

The threshold obtained from the mouse sample to differentiate the tumor and healthy domains were applied. The ratio mappings (FIG. 8C) indicate that all the area of the healthy tissue exhibit CH_{S}/CH_{2,Ss} and NH_{S}/CH_{2,Ss} ratio smaller than the threshold, i.e. 0.22, and 8.5, respectively, while the tumor tissues show spatial dependent ratio with the majority of domains above the threshold (shown in FIG. 8C) and a few exception areas. Similar to the mouse tissue, the areas identified as tumor tissues using CH_{S}/CH_{2,Ss} and NH_{S}/CH_{2,Ss} ratios largely agree with each other. It is noted that a similar tumor identification result was obtained if applying a new threshold obtained from statistical analyzing an area of the healthy human lung tissue. Thus, the spectral peak ratio of VSFG reflects fundamental structural changes in collagens so that the method demonstrated here are not specific to a particular host species.

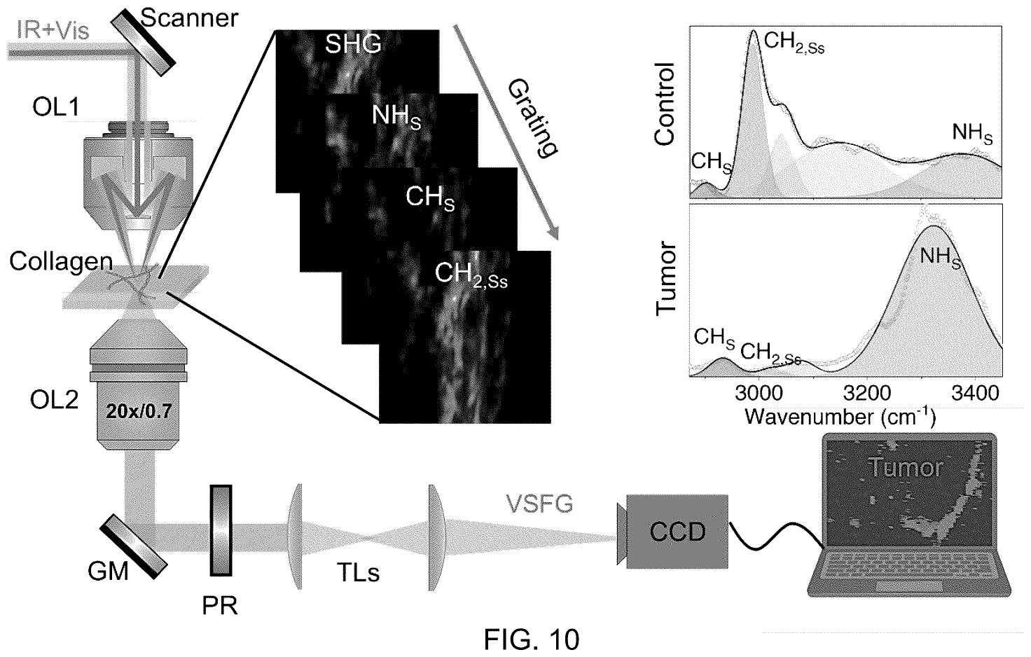

The disclosed method provides that VSFG microscopy platform could directly image tumor domain based on the NH_{S}/CH_{2,Ss} and CH_{S}/CH_{2,Ss} ratio mapping. The image signature profiles can be analyzed for tumor domain details of collagen. As shown in FIG. 10, the VSFG microscope setup for detecting tumor related collagen includes two incident optical beams: one infrared (IR) beam at ω_{IR} and an up-conversion beam at ω_{VIS}, which generate a signal at the sum frequency ω_{SFG}=ω_{IR}+ω_{VIS}. The NIR and MIR beams are focused onto the collagen sample using Objective 1 (OL1). After turning on the resonant beam scanner to raster the laser beams on the tissue samples, the resulting VSFG signal is collected using Objective 2 (OL2). The VSFG signal is then directed to the spectrometer and CCD for analysis. This setup allows to obtain VSFG hyperspectral images, meaning VSFG spectral images at each pixel. By analyzing the spectral signatures, specifically the NH_{S}/CH₂, ss ratio and CH_{S}/CH₂, ss ratio, it will directly differentiate between tumor and healthy tissues.

### Comparison to Other Optical Methods

It was further examined whether similar results could be obtained by SHG images (FIG. 8A) and VSFG spectroscopy. The overall intensity of SHG was lower than that of VSFG, likely because SHG involved only non-resonant excitations, unlike VSFG. Furthermore, SHG only revealed the morphological and intensity differences between the two samples, which alone cannot differentiate tumor tissues from healthy ones.

For VSFG spectroscopy (without the imaging modality), although the spectra of the tumor and control samples display a similar line shape to those obtained using VSFG microscopy, the signal-to-noise ratio was extremely low under the same data acquisition condition, prohibiting quantitative analysis. This could be due to the ensemble-averaging of many fiber domains, which destructively interfere with each other due to the random orientations.

While VSFG shows marked spectral differences between the collagen tissues bearing metastatic tumors and healthy controls, exploring the origins of these differences is crucial. One potential reason is that the CH_{2,Ss} modes and NH_{S} (and CH_{S}) modes have different orientations, leading to the observed intensity change due to the relative orientation between the sample and laser beam polarization. If this were the case, it would be expected the relative peak intensity to vary dramatically as the polarization combination of VSFG pulse sequence changes. However, it was observed that all spectra from the same sample species display similar relative spectral peak intensities regardless of the polarization schemes. Thus, it can be ruled out that the peak ratios changes are due to sample orientations.

This leaves the origin of the peak ratio change to be structural, either on the molecular level, or on the mesoscopic assembly level. We first consider whether large chemical modifications occur, such as changes in amino acid structures through crossing-linking among peptide chains. If large chemical modifications occurred, they would likely affect the amide bonds. However, in the OPTIR hyperspectral imaging, there were no dramatic spectral changes observed in the N—H modes (Amide A) between the tumor and control tissues. This leaves substantial chemical structure changes unlikely.

### Theoretical Models

It was further evaluated how collagen packing may result in the change of observed VSFG spectra. There could be several possible changes: (i) relative fibril alignments, (ii) interfibrillar distances, and (iii) fibril diameters. We can rule out the first one because if fibril alignments change from ordered to random, the symmetry of collagen samples would be lost, leading to a decrease of the overall VSFG signal. The similar spectral peak ratio regardless of the polarization schemes further confirm our interpretation.

To explore options (ii) and (iii), it was applied the established nonlinear optical theory of VSFG to determine their influence on the coherent signal. The dependence of the VSFG intensity of uniaxially aligned collagen fibrils on the fibril diameter (d) and the distance between collagen fibrils (Δl) can be modeled as the following.

$${I}_{VSFG} ∝ {I}_{VIS} {I}_{IR} {\vert {χ}_{IJK}^{( 2 )} \vert}^{2} \qquad ( 1 )$$

$${χ}_{IJK}^{( 2 )} = {\sum }_{IJK} < {R}_{Ii} {R}_{Jj} {R}_{Kk} > {\sum }_{f = 0}^{n - 1} {β}_{a}^{fbl} \frac{{e}^{- i Δ kd} - 1}{Δ k} ( {e}^{- i Δ k ( d + Δ l ) f} ) n ( {( - 1 )}^{f} ( cos α ) + cos γsin α ) \qquad ( 2 )$$

Here, I_{VSFG}, I_{VIS}, and I_{IR} represent the intensities of the VSFG signal, the 1030 nm up-conversion beam, and the resonant IR beam, respectively.

$${χ}_{IJK}^{( 2 )}$$

is the effective second-order susceptibility tensor of the VSFG-active vibrational mode of the fibril, corresponding to the specific polarization combinations of VSFG, 1030 nm, and IR beams. R_{Ii}, R_{Jj} and R_{Kk} are the Euler matrices transforming the fiber frame to the lab frame.

$${β}_{a}^{fbl}$$

are the hyperpolarizability of the fibril frame. The phase mismatch is Δk=k_{IR}+kᵥᵢₛ−k_{VSFG}, a is the angle of the transition dipole vector with respect to the c axis, γ is the angle of the transition dipole vector with respect to the b axis (illustrated in FIG. 6A), and n is the number of crystals within the coherence length (L_{c}), n=L_{c}/(d+Δl), where the coherent length

$${L}_{c} = \frac{2}{Δ k} ,$$

d is the diameter of the collagen fibril and Δl is the effective spacing between the fibrils. d+Δl can be regarded as the interfibrillar distance, which influences the coherent sum of the VSFG signals. The (e^{-iΔk(d+Δl)f}) term represents the quasi-random phase-matching process by the neighboring collagen fibrils. The

$$\frac{{e}^{- i Δ kd} - 1}{Δ k}$$

term accounts for the phase matching synchronization of the VSFG signal. The (−1)^{f}(cos α)+cos γ sin α) term represents the bidirectional packing of the collagen fibrils.

To simulate the VSFG intensity, it was firstly determined the effective second-order susceptibility tensor of fibril,

$${χ}_{IJK}^{( 2 )} .$$

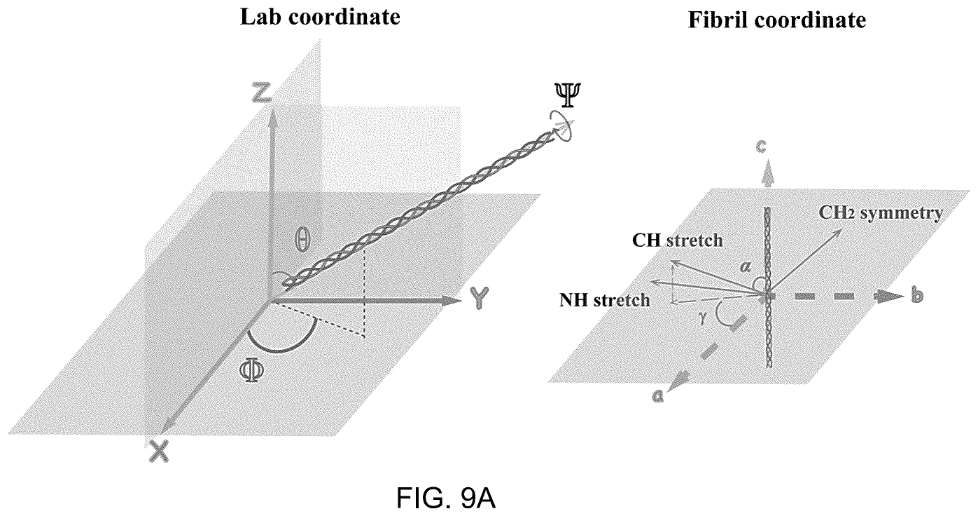

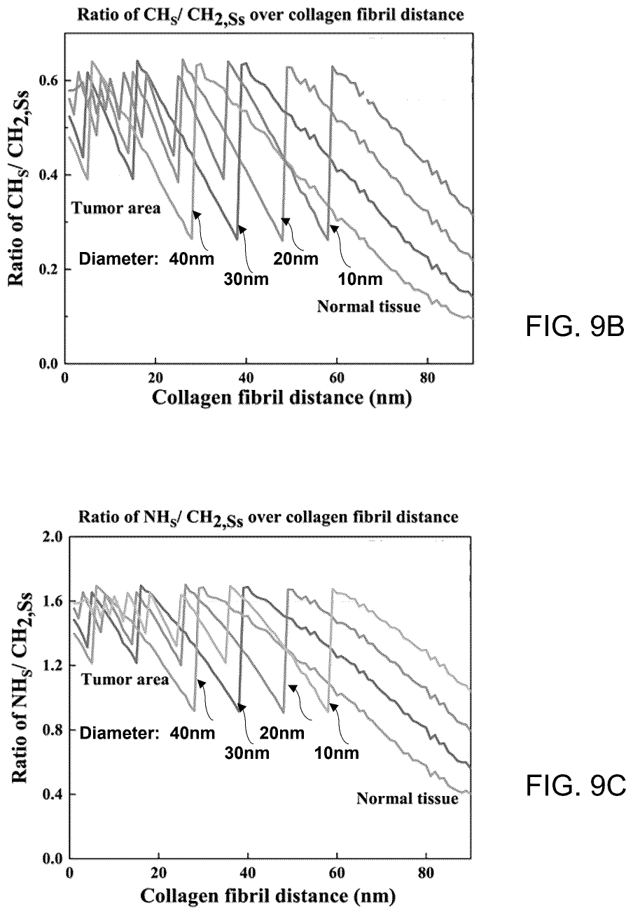

The computation was made on hyperpolarizability for each amino acid on the collagen I structure using Density Functional Theory (DFT) calculation with B3LYP/6-31g (d) level of theory. By taking the sum of the hyperpolarizability tensor of amino acid, we can obtain second order susceptibilities of the fibril. This methodology aligns with the vibrational mode calculation of biological macromolecules in previous study. The calculated angles of the vibrational modes with respect to the triple helix direction (c-axis) are as follows: CH_{S} (60°), CH_{2,Ss} (45°), and NH_{S} (80°), as shown in FIG. 9A. Subsequently, the fiber 2^{nd} order susceptibility is calculated using equation (2) by summing contributions of individual fibrils which takes into account the interfibrillar distance and fibril diameter.

FIGS. 9B-9C show the impact of collagen fibril distance on the ratios of CH_{S} to CH_{2Ss} mode (left plot) and NH_{S} stretch to CH_{2Ss} mode (right plot) for various collagen fibril diameters. Both the CH_{S}/CH_{2Ss} and NH_{S}/CH_{2Ss} intensity decrease with increasing fibril distance with a certain range, across all collagen fibril diameters. The NH_{S} intensities become smaller than the CH_{2,Ss} intensity as the fibril distance decreases. This result suggests that the different vibrational mode interferes in their own way due to the different effective second-order susceptibilities. This makes senses as the NH_{S} and CH_{S} modes have similar orientation while the CH_{2,Ss} orients differently in the fibril frame. The oscillatory features shown in FIGS. 9B-9C are caused by two effects: (i) As the inter-fibrillar distance Δl increases, the quasi-random phase matching term (e^{-iΔk(d+Δl)f}) undergoes periodic modulation. (ii) In the bidirectional packing, the odd-numbered (even-numbered) collagen fibrils correspond to intensity maxima (minima). This is due to the symmetry cancellation effect of transition dipoles oriented in opposite directions. The interplay between these two mechanisms results in an oscillation behavior.

Considering that the simulation results suggested a decrease of interfibrillar effective spacings within a certain range, by reduced distance, enlarged fibril diameters or both, corresponding to an increase in the packing density, this insight remarkably agrees with our mechanical measurements from AFM. The AFM results indicate that the tumor-related collagen is stiffer than healthy collagen. As the elastic modulus has positive relation with the density, these two results now corroborate with each other—the collagen tissues in the tumor environments engendered a denser packing arrangement, leading to enhanced stiffness. These preliminary structural insights warrant further quantitative theoretical analysis and investigation using other high-level structural biology techniques, such as cryo-transmission electron microscopy in the future.

The inventive VSFG imaging techniques combine the best of both worlds from coherent SHG and IR absorption spectroscopy. By using vibrational modes as the coherent light emitting source, different vibrational modes in the sample interfere distinctly, resulting in unique spectral line shapes that can identify collagens in tumor samples versus healthy tissues. The inventive approach provides a new label-free method to detect tumor tissues. By combining fiber optics and endoscope techniques, the inventive approach provides a new non-invasive in vivo diagnostic tool.

Combining VSFG imaging with theoretical simulations, it is corroborated that the spectral alternation is due to the collagen density change, resulting in the effective spacing between adjacent fibrils being smaller than the coherence length. The results align with the heightened stiffness in the tumor collagen samples, offering structural insights to the widely observed mechanical property changes of collagen in the tumor samples.

Although the inventive platform and techniques have been described in considerable detail with reference to certain preferred embodiments and examples, other modifications and implementations may become apparent to those of skill in the art based on the concepts and teachings provided herein. Accordingly, the scope of the appended claims should not be limited by the foregoing disclosure and description of preferred embodiments but should be construed to include obvious variations.

What is claimed is:

1. A multimodal hyperspectral VSFG microscope platform, comprising:
a pulsed laser beam;
an optical parametric amplifier (OPA), configured to guide the output of the laser beam, generating a mid-infrared (MIR) beam;
a Fabri-Perot etalon, configured to pass the residue laser beam from the OPA for an up-conversion near IR (NIR) beam;
a dichroic mirror (DM), wherein the MIR beam and the NIR beam are spatially overlapped;
a resonant beam scanner mounted to an integrated 2-position slider (I2PS);
an inverted microscope configured to integrate with the beam scanner, wherein the MIR beam and the NIR beam are overlapped and are directed into the inverted microscope;
a reflective-based infinity-corrected Schwarzschild objective (SO) and a refractive-based infinity-corrected imaging objective (RO) mounted to a vertical nanopositioning (VNP) z-axis stage;
a microscope stage for retaining a sample, mounted between the SO and the RO, whereby overlapped beams from SO are focused onto the sample and a VSFG signal generated by the sample is collected by RO;
a linear polarizer and a telecentric tube lens system, whereby the signal is guided through and processed;
a monochromator (MC), wherein a magnified image formed at the entrance slit of the MC; and
a charge-coupled device (CCD) coupled with the linear polarizer and the telecentric tube lens system, whereby a magnified image and data are detected and collected.

2. The platform of claim 1, wherein the laser beam having ˜100 fs time duration;

3. The platform of claim 1, wherein the DM is transmissive to the MIR and reflective to the NIR.

4. The platform of claim 1, wherein the SO is purely reflective, acting as a condenser.

5. The platform of claim 1, wherein the z-axis position of the RO is controlled at 1 μm precision.

6. The platform of claim 1, wherein the tube lens system comprises two tub lenses (TL1 and TL2).

7. A method for detecting tumor in a biological tissue sample with the VSFG microscope platform of claim 1, comprising:
mounting the tissue sample on a slide to the microscope stage;
subjecting the tissue sample with NIR and MIR beams using Objective 1 (OL1) of the microscope;
turning on the resonant beam scanner to raster the laser beams on the tissue samples;
collecting a resulting VSFG signal using Objective 2 (OL2) of the microscope;
directing the VSFG signal to a spectrometer and a CCD; and
obtaining VSFG hyperspectral images, whereby the tumor is detected.

8. The method of claim 7, wherein the biological tissue sample comprises collagen.

9. The method of claim 7, wherein the biological tissue sample is derived from lung tumor, breast cancer, colorectal liver metastases, esophageal squamous cell carcinoma, or malignant ovarian neoplasms.

10. A method for detecting tumor in a biological tissue sample with the VSFG microscope platform of claim 1, comprising:
mounting the tissue sample on a slide of the microscope stage;
subjecting the tissue sample with NIR and MIR beams using Objective 1 (OL1) of the microscope;
turning on the resonant beam scanner to raster the laser beams on the tissue sample;
collecting a resulting VSFG signal using Objective 2 (OL2) of the microscope;
directing the VSFG signal to a spectrometer and a CCD;
obtaining VSFG hyperspectral images; and
analyzing spectral signatures of the images for a NH_{S}/CH_{2,Ss} and
CH_{S}/CH_{2,Ss} ratio mapping wherein the tumor is detected.

11. The method of claim 10, wherein the biological tissue sample comprises collagen.

12. The method of claim 10, wherein the biological tissue sample is derived from lung tumor, breast cancer, colorectal liver metastases, esophageal squamous cell carcinoma, or malignant ovarian neoplasms.

13. The method of claim 12, wherein the biological tissue sample is derived from lung tumor.
